(19) United States
(12) Patent Application Publication
(10) Pub. No.: US 2006/0234948 A1
(43) Pub. Date: Oct. 19, 2006

(54) Lignan-containing compositions

(76) Inventors: Mark W. Empie, Forsyth, IL (US); Brent D. Flickinger, Decatur, IL (US); Sam Z. Sun, Decatur, IL (US)

Correspondence Address: KIRKPATRICK & LOCKHART NICHOLSON GRAHAM LLP;HENRY W. OLIVER BUILDING, 535 SMITHFIELD STREET, PITTSBURGH, PA, 15222, US

(21) Appl. No.: 11/397,239

(22) Filed: Apr. 3, 2006

Related U.S. Application Data
(60) Provisional application No. 60/667,937, filed on Apr. 4, 2005.

(51) Int. Cl. A61K 31/7048; A61K 31/7008; A61K 31/355; A61K 31/353; A61K 31/12

(52) U.S. Cl. 514/22; 514/27; 514/62; 514/456; 514/458; 514/171; 514/690

(57) ABSTRACT

Compositions are provided that includes a lignan, and an additional compound such as an isoflavone, a tocopherol, a phytosterol, a polyphenol, a catechin, an anthocyanin, an astaxanthin, or a glucosamine. The compositions can be formulated as a dietary supplement, in tablet, powder or liquid form, or can be incorporated into a food product. Methods of treating various diseases by administering the compositions are also provided.

### CROSS-REFERENCE TO RELATED APPLICATIONS

This application claims the benefit of U.S. Provisional Application No. 60/667,937, filed on Apr. 4, 2005. The entire teachings of that application are incorporated herein by reference.

### FIELD

The present invention relates to compositions including a lignan, for example a flax lignan, and another plant secondary metabolite, such as, for example, an isoflavone, a phytosterol, an astaxanthin, a tocopherol, a catechin, a polyphenol, an anthocyanin, or a glucosamine.

### BACKGROUND

Epidemiology studies relating diet to disease suggest that dietary components may cause a reduced risk of certain diseases in some populations. For instance, far eastern populations consuming soy as a staple have reduced rates of breast, colon and prostate cancers and coronary heart disease, while populations in Finland have reduced rates of prostate cancer. Researchers are presently studying specific dietary compounds in order to understand the basis for these epidemiological observations.

Several plants consumed in the diet are rich sources of beneficial phytochemicals. Soy products contain high concentrations of isoflavones and saponins. Unrefined grains including, without limitation, wheat, psyllium, rice, flax and oats, all contain lignans, which are compounds closely related to lignins. Cocoa contains phenolic acids and catechins, which also occur in green teas. Certain non-dietary plants are also sources of these same chemical molecules, such as lignans and isoflavones in kudzu root and red clovers. Isoflavones and lignans act as weak estrogenic substances. Tea plants are also a rich source of phytochemicals, including catechins and phenolic acids.

Numerous reports in literature have documented the phytochemical benefits of flaxseed lignans. Rickard et al. reported that feeding purified lignan at 5% flaxseed diet levels significantly reduces colon and mammary carcinogenesis in animals (Proceedings of the 57th Flax Institute of the United States, (Fargo, N.Dak.):8-13 (1998)). Demark-Wahnefried et al. also reported that flaxseed supplementation may have a beneficial effect on prostate cancer biology (Demark-Wahnefried et al., Adult Urology 58(1):47-52 (2001)).

Additionally, it has been reported that lignans reduce the incidence of Type I and Type II diabetes by 71% (Prasad, K., Proc. of the American Diabetes Association (1999)), act as a hypotensive agent, lowering blood pressure without affecting heart rate (U.S. patent application Ser. No. 60/140,972, filed Jun. 16, 1999), provide benefits against Lupus Nephritis (U.S. Pat. No. 5,827,256), and reduce development of hypercholesterolemic atherosclerosis in animals (Atherosclerosis 132:69-76(1997)). In addition, there have been numerous reports of potential antioxidant (Mol. & Cell. Biochem. 202:91-100 (1999)) and anticancer properties (Anticancer Research 18:1405-1408 (1998)) of lignans.

Flaxseed, in whole, ground, or defatted form has been incorporated into animal feeds and food products such as breads, cookies, bagels and muffins. It has also been used for supplementing fiber levels in meat products (see, e.g., WO 00/19842).

### SUMMARY

Purified compositions are provided including a lignan, preferably a lignan extracted from flax, and an additional compound such as an isoflavone, a tocopherol, a phytosterol, a polyphenol, a catechin, an anthocyanin, an astaxanthin, or a glucosamine. The compositions can be formulated as a dietary supplement, in dosage form such as tablets, powder, or liquid form. The compositions can be used in food, snack or beverage products. The compositions can be in any form suitable for use in supplementing the diet.

Also provided are methods of administering the compositions disclosed herein to treat ailments, conditions, diseases, or address symptoms thereof. For instance, the compositions can be used to treat heart disease (by alleviating symptoms associated with heart disease, preventing hypercholesterolemic atherosclerosis, reducing the risk of heart disease, lowering serum cholesterol, reducing ischemic damage and increasing nitric oxide expression in endothelial cells); diabetes (by alleviating symptoms associated with diabetes, preventing or delaying the onset of diabetes, and modulating serum glucose levels); osteoporosis and arthritis (by increasing bone density, reducing loss of bone density with increasing age, and alleviating symptoms associates with arthritis); prostate cancer (by reducing the risk of prostate cancer, and delaying the onset of prostate cancer); and gynecological conditions (by alleviating symptoms associated with the onset of menstruation, and alleviating symptoms associated with the onset of menopause). The compositions also can be used to inhibit cyclooxygenase activity in macrophages and endothelial cells. The compositions also can be used to prevent or delay metastasis.

Methods are also provided for using the compositions disclosed herein in treating a disease, wherein the disease can be assayed by use of a serum-specific marker. The methods include assaying the serum-specific marker in a person thought to have the disease, administering the composition to the person, and re-assaying the serum-specific marker. Reduction in the amount of the serum-specific marker in the second assay relative to the first indicates that the compound is effectively treating the disease.

A composition is provided that comprises a purified lignan, for example a flax lignan, and an additional purified compound, which can be an isoflavone, for example a soy isoflavone, a tocopherol, for example a γ-tocopherol, a phytosterol, a polyphenol, a catechin, an anthocyanin, an astaxanthin or a glucosamine, for example glucosamine sulfate. The lignan can be present in an amount that is about 4 times to about 6 times the amount of isoflavone by weight present in the composition, about 2 times to about 6 times the amount of tocopherol by weight present in the composition, about 1 times to about 2 times the amount of phytosterol by weight present in the composition, about 1 times to about 2 times the amount of polyphenol by weight present in the composition, about 1 times to about 6 times the amount of catechin by weight present in the composition, about 2 times to about 6 times the amount of anthocyanin by weight present in the composition, about 100 times to about 400 times the amount of astaxanthin by weight present in the composition, or about one-fifth the amount of glucosamine in the composition. The composition can be in the form of a tablet, a powder or a liquid.

Also provided is a food product containing the composition. Any food product potentially can be formulated to contain the composition. The food product can be a single-serving food product. The food product can include about 100 mg to about 500 mg of lignan. The food product also can include about 15 mg to about 120 mg isoflavone, about 15 mg to about 200 mg of tocopherol, about 50 mg to about 800 mg phytosterol, about 50 mg to about 500 mg polyphenol, about 15 mg to about 300 mg catechin, about 15 mg to about 200 mg anthocyanin, about 0.25 mg to about 5 mg astaxanthin, or about 0.5 grams to about 2 grams glucosamine.

Also provided is a dietary supplement that comprises a purified lignan, for example a flax lignan, and an additional purified compound, which can be an isoflavone, for example a soy isoflavone, a tocopherol, for example a γ-tocopherol, a phytosterol, a polyphenol, a catechin, an anthocyanin, an astaxanthin or a glucosamine, for example glucosamine sulfate. The dietary supplement can be in the form of a tablet, a powder or a liquid. The dietary supplement in tablet form can include about 100 mg to about 500 mg of lignan. The dietary supplement in tablet form also can include about 15 mg to about 120 mg isoflavone, about 15 mg to about 200 mg of tocopherol, about 50 mg to about 400 mg phytosterol, about 50 mg to about 500 mg polyphenol, about 15 mg to about 300 mg catechin, about 15 mg to about 200 mg anthocyanin, about 0.25 mg to about 5 mg astaxanthin, or about 0.5 grams to about 2 grams glucosamine.

The invention also provides a method of treating a human, where the method includes administering to the human a composition as described herein, where: if the treatment is for alleviating symptoms associated with heart disease, the composition comprises a purified lignan and a purified compound selected from the group consisting of: an isoflavone, a tocopherol, a phytosterol, a polyphenol, a catechin, an anthocyanin and an astaxanthin; if the treatment is for preventing hypercholesterolemic atherosclerosis, the composition comprises a purified lignan and a purified compound selected from the group consisting of: an isoflavone, a tocopherol, a phytosterol, a polyphenol, a catechin, an anthocyanin and an astaxanthin; if the treatment is for reducing the risk of heart disease, the composition comprises a purified lignan and an additional purified compound selected from the group consisting of: an isoflavone, a tocopherol, a phytosterol, a polyphenol, a catechin, an anthocyanin and an astaxanthin; if the treatment is for lowering serum cholesterol, the composition comprises a purified lignan and an additional purified compound selected from the group consisting of: an isoflavone, a tocopherol, a phytosterol, a polyphenol, a catechin, an anthocyanin and an astaxanthin; if the treatment is for reducing ischemic damage, the composition comprises a purified lignan and an additional purified compound selected from the group consisting of: an isoflavone, a tocopherol, a phytosterol, a polyphenol, a catechin, an anthocyanin and an astaxanthin; if the treatment is for increasing nitric oxide expression in endothelial cells, the composition comprises a purified lignan and an additional purified compound selected from the group consisting of: an isoflavone, a tocopherol, a phytosterol, a polyphenol, a catechin, an anthocyanin and an astaxanthin; if the treatment is for inhibiting cyclooxygenase activity in macrophages, the composition comprises a purified lignan and a purified tocopherol; if the treatment is for inhibiting cyclooxygenase activity in endothelial cells, the composition comprises a purified lignan and a purified tocopherol; if the treatment is for alleviating symptoms associated with diabetes, the composition comprises a purified lignan and an additional purified compound selected from the group consisting of: an isoflavone, a tocopherol, a polyphenol, a catechin, an anthocyanin and an astaxanthin; if the treatment is for preventing or delaying the onset of diabetes, the composition comprises a purified lignan and an additional purified compound selected from the group consisting of: an isoflavone, a tocopherol, a polyphenol, a catechin, an anthocyanin and an astaxanthin; if the treatment is for modulating serum glucose levels, the composition comprises a purified lignan and an additional purified compound selected from the group consisting of: an isoflavone, a tocopherol, a polyphenol, a catechin, an anthocyanin and an astaxanthin; if the treatment is for increasing bone density, the composition comprises a purified lignan and an additional purified compound selected from the group consisting of: an isoflavone and a glucosamine; if the treatment is for reducing loss of bone density with increasing age, the composition comprises a purified lignan and an additional purified compound selected from the group consisting of: an isoflavone and a glucosamine; if the treatment is for alleviating symptoms associates with arthritis, the composition comprises a purified lignan and an additional purified compound selected from the group consisting of: an isoflavone and a glucosamine; if the treatment is for reducing the risk of prostate cancer, the composition comprises a purified lignan and an additional purified compound selected from the group consisting of: an isoflavone, a tocopherol, a phytosterol, a polyphenol, a catechin, an anthocyanin and an astaxanthin; if the treatment is for delaying the onset of prostate cancer, the composition comprises a purified lignan and an additional purified compound selected from the group consisting of: an isoflavone, a tocopherol, a phytosterol, a polyphenol, a catechin, an anthocyanin and an astaxanthin; if the treatment is for reducing the risk of metastasis, the composition comprises a purified lignan and an additional purified compound selected from the group consisting of: an isoflavone, a tocopherol, a phytosterol, a polyphenol, a catechin, an anthocyanin, an astaxanthin and glucosamine; if the treatment is for delaying metastasis, the composition comprises a purified lignan and an additional purified compound selected from the group consisting of: an isoflavone, a tocopherol, a phytosterol, a polyphenol, a catechin, an anthocyanin, an astaxanthin and glucosamine; if the treatment is for alleviating symptoms associated with the onset of menstruation, the composition comprises a purified lignan and a purified isoflavone; or if the treatment is for alleviating symptoms associated with the onset of menopause, the composition comprises a purified lignan and a purified isoflavone.

Also provided is a method of treating a disease or condition, where the method includes: assaying a marker specific for the disease or condition in a subject thought to have the disease or condition; administering the composition of claim 1 to the subject; and assaying the marker in the subject again; where a change in the amount of the marker assayed indicates that the disease or condition is being treated.

### DETAILED DESCRIPTION

Other than in the examples described herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

In one aspect, purified compositions are provided that include a lignan, preferably a flax lignan, and an additional compound. The additional compound can be an isoflavone, a tocopherol, a phytosterol, a polyphenol, a catechin, an anthocyanin, an astaxanthin, or a glucosamine. In another aspect, the compositions are provided in the form of a dietary supplement, in tablet, powder or liquid form. The compositions can also be formulated to be included in any food composition.

Also provided are methods of administering the compositions, dietary supplements and food products as described herein to treat ailments, conditions, diseases or symptoms thereof. For instance, the compositions can be used to treat heart disease, for example, the compositions can be used to alleviate symptoms associated with heart disease, prevent hypercholesterolemic atherosclerosis, reduce the risk of heart disease, lower serum cholesterol, reduce ischemic damage and increase nitric oxide expression in endothelial cells. The compositions also can be used to alleviate symptoms associated with diabetes, prevent or delay the onset of diabetes, and modulate serum glucose levels. The compositions can also be used to prevent, reduce the risk of, or delay metastasis. The compositions can be administered to increase bone density, reduce loss of bone density with increasing age, alleviate symptoms associates with arthritis, reduce the risk or delay the onset of prostate cancer, alleviate symptoms associated with the onset of menstruation, alleviate symptoms associated with the onset of menopause, and inhibit cyclooxygenase activity in macrophages and endothelial cells.

Many secondary plant metabolites have been found to have beneficial health properties. Usually these properties have been discovered initially by epidemiological studies showing reduced disease incidence in populations with diets high in particular plant components. Asian populations that consume large amounts of soy, for instance, have a lower incidence of certain cancers than Western populations.

A number of plant secondary metabolites for which there is evidence of health benefits are listed below.

Lignans

Lignans are secondary plant metabolites that are produced from shikimic acid via the phenylpropanoid pathway. Lignans develop from flavonoid precursors and aid in protecting plants from certain pathogens and predators. Lignans are defined as compounds possessing a 2,3-dibenzylbutane structure and include matairesinol, secoisolaricinesinol, lariuresinol, isolariciresinol, nordihydroguaiaretic acid, pinoresinol, olivil and other compounds, and modifications thereof, including diglucosides such as but not limited to herbacetin 3,8-0-diglucopyranoside, herbacetin 3,7-0-dimethyl ether and Kaempferol 3,7-0-diglucopyranoside. Diglycerides are known precursors of two important mammalian lignans dibenzylbutyrolactone enterolactone and dibenzyl butane enterodiol (Setchell et al., Biochem. J. 197:447-458 (1981)).

Flaxseed (Linum usitatissimum) is potentially the richest source of phytoestrogens including lignans. The primary lignan found in flaxseed is 2,3-bis (3-methoxy-4-hydroxybenzyl) butane-1,4-diol (secoisolariciresinol) which is stored as the conjugate secoisolariciresinol diglucoside (SDG) in its native state in the plant. Flax seed contains levels of these phytoestrogens that are 75-800 times greater than any other plant food. The plant lignan, catecholic nordihydroguaiaretic acid, is a potent antioxidant previously used by the food industry. The flax lignans can also be used in the form of isolated fiber, pressed oil, or extracted, for instance, according to the process disclosed in U.S. Pat. No. 6,767,565. The lignans may also be obtained from the process described in U.S. Provisional Patent Application 60/742,082, filed Dec. 2, 2005, the contents of the entirety of which are incorporated herein by this reference.

Isoflavones

It has been proposed to use isoflavones to treat or prevent breast cancer, prostate cancer, skin cancer and colon cancer. It also has been proposed that use of isoflavones may reduce or prevent various symptoms related to the onset and duration of menopause, including hot flashes and osteoporosis, and may be effective in certain cardiovascular applications, including prevention of heart disease, reducing cholesterol-lipid levels, modulating angiogenesis, and providing other advantageous vascular effects. Moreover, isoflavones have been implicated in reducing headaches, dementia and inflammation, treating alcohol abuse, and may play a role in immunomodulation.

Isoflavones, which are sometimes referred to as phytoestrogens, are heterocyclic phenols. Soy and other plants such as red clover contain a variety of beneficial isoflavones. Non-limiting examples of specific isoflavones found in soy protein include: the glucosides such as genistin, daidzin and glycitin, and the de-methylated aglycone forms such as genistein, daidzein and glycitein. Red clover contains these isoflavones, along with the isoflavones biochanin A and formononetin. These compounds are believed to have similar activities once they are ingested. Ingestion of soy protein specifically has been shown to significantly reduce menopausal symptoms such as “hot flashes,” although not to the extent of conventional estrogen replacement therapy. Soy protein also appears to be useful in treating cyclical mastalgia (breast pain associated with the menstrual cycle). Soy isoflavones are also antioxidants and increase bone density, and soy protein containing high levels of isoflavones has been shown to have a lipid-lowering effect. Although conventional pharmaceuticals can be used to obtain all of these effects, soy protein is often used by those seeking a “natural” alternative to hormone replacement therapy, calcium supplements, and cholesterol-lowering drugs, and as an aid to preventing cancer.

People eating a high-soy diet show a reduction of many of the above-discussed symptoms. Thus, ingesting a combination of certain phytochemicals in a ratio such as found in soy may result in an additive or synergistic effect. However, a high soy diet also may have certain undesirable effects, including flatulence and undesirable taste, and Western consumers are characteristically hesitant to change their lifestyle to incorporate soy in their diets, despite the foregoing health benefits.

Tocopherols

Tocopherols are antioxidants and constitute the different forms of vitamin E. There are four principal homologues of tocopherols, namely, alpha, beta, gamma and delta tocopherols. Although these four homologues of vitamin E exhibit biological activity, α-tocopherol possesses the highest biological activity.

Tocopherols can be found in various ratios and concentrations in crude vegetable oils such as soybean, sunflower, canola, rapeseed, cottonseed, palm oil, and rice bran oil. Tocopherols are valuable constituents of vegetable oil and have a number of practical applications. For example, tocopherols help prevent oxidation and spoilage of food, and are also valuable dietary supplements because they can reduce the risk of certain types of cancer.

As a class of compounds, the tocopherols have been extensively studied. Through the findings of these studies, certain biological activities have been attributed to the tocopherols. Most notably, tocopherols possess strong antioxidant properties. Tocopherols exhibit a strong ability to remove free radicals. Because of this characteristic, tocopherols are included in a wide variety of consumer products, including foodstuffs and cosmetics.

Phytosterols

Phytosterols are plant sterols structurally similar to cholesterol that for many years have been to reduce cholesterol absorption and serum cholesterol levels, while not being absorbed in the intestinal tract. Phytosterols are therefore useful in treating individuals with mildly increased serum cholesterol, and are included in food products and dietary supplements sold to the general population.

Chemically, naturally occurring sterols are C₂₆-C₃₀ steroid alcohols having an aliphatic side chain at the C₁₇ position. The structural differences between a cholesterol molecule and a phytosterol molecule are primarily found in the structure of the side chain of the basic frame. Plant sterols may also be hydrogenated to produce plant stanols, i.e., phytostanols.

Polyphenols

Plant phenolic compounds occur as free monomers or in combination with other phytochemicals, thereby forming esters or glycosides. Phenolic acids are known to have antioxidant activity. The major phenolic constituents of flaxseed are reported to be cournaric acid (4-glucosyl-cinnamic acid), caffeic acid (3-hydroxy-4-glucosyl-cinnamic acid), ferulic acid (3-methoxy-4glucosyl cinnamic acid) and hydroxy methyl glutamic acid. These compounds have antioxidant and hyperchlolesteremic properties.

Polyphenols are found in cocoa, coffee, tea, and other theobroma species. Polyphenols exhibit antioxidant activity and include catechins, epicatechins, and procyanidins. Procyanidins are polymeric polyphenols that include catechins as basic structural elements. Polyphenols have an inhibitory effect on mutagenesis and carcinogenesis. Polyphenols are believed to be active in preventing cancer, coronary and cardiovascular disease and strokes, periodontal disease, atherosclerosis and hypertension; inhibit LDL oxidation and DNA topoisomerase IT; and modulate cyclo-oxygenase, lipoxygenase, nitric oxide or NO-synthase, apoptosis and platelet aggregation. They also possess anti-inflammatory activity and have been implicated in delaying aging processes. These antioxidants are usually water-soluble and comprise compounds of the chroman type, such as catechin and epicatechin (the stereoisomeric 2-(3,4-dihydroxyphenyl)-3,5,7-trihydroxychromans), and oligomerised structures including procyanidin.

Catechins

Catechins, or flavan-3-ols, are a type of polyphenol that appears in green tea, black tea, grapes, wine, and chocolate. Polyphenol catechins in green tea include gallocatechin (GC), epigallocatechin (EGC), epicatechin (EC) and epigallocatechin gallate (EGCG). They have potent antioxidant capabilities and are being studied for their ability to prevent cancer and heart disease.

Anthocyanins

Anthocyanins are a large class of naturally occurring pigments that provide color to fruits and vegetables. They belong to the flavenoid class of plant compounds, and flavenoids are in turn are a subclass of plant polyphenol. Anthocyanins have antioxidant activity, and are being studied for their anticarcinogenic activity, and their ability to reduce LDL cholesterol levels and prevent blood clotting.

Astaxanthin

Astaxanthin is a carotenoid pigment that occurs naturally in shellfish and algae. It is a powerful biological antioxidant, exhibits strong free radical scavenging activity and protects against lipid peroxidation and oxidative damage of LDL-cholesterol, cell membranes, cells and tissues. Its possible role in cardiovascular diseases, eye health, neurodegenerative diseases, aging, immune responses, oxidative stress and cancer are under study.

Carotenoids are naturally-occurring yellow to red pigments of the terpenoid group that can be found in plants, algae, bacteria, and certain animals, such as birds and shellfish. Carotenoids include hydrocarbons (carotenes) and their oxygenated, alcoholic derivatives (xanthophylls). Carotenoids include actinioerythrol, astaxanthin, bixin, canthaxanthin, capsanthin, capsorubin, β-8′-apo-carotenal (apo-carotenal), β-12′-apo-carotenal, a-carotene, β-carotene, “carotene” (a mixture of α- and β-carotenes), γ-carotene, β-cryptoxanthin, lutein, lycopene, violerythrin, zeaxanthin and esters of hydroxyl- or carboxyl-containing members thereof. Many of the carotenoids occur in nature as cis- and trans-isomeric forms, while synthetic compounds are frequently racemic mixtures.

Carotenoids are biologically active, and are used in the food, pharmaceutical and nutritional supplement fields because of their health benefits. Carotenoids have been studied for their effect on age-related macular degeneration, skin cancer and heart disease, and lycopene has been linked to reduced risk of heart disease and prostate cancer.

Glucosamine

Glucosamine is a sugar produced in the body and maintains cartilage. It is found in small amounts in the shells of shellfish, and it is generally taken as a dietary supplement to help relieve the pain and stiffness of arthritis and other degenerative joint disorders. Supplement forms include glucosamine sulfate, glucosamine hydrochloride and N-acetyl-glucosamine.

The above compounds can be ingested by consumption of plant material, but the amount will be limited by the quantity of plant material that can be consumed by an individual. The compounds can be extracted, concentrated and purified, but then must be processed to a form suitable for direct consumption by the individual (e.g., as tablets, powders or liquids, as for dietary supplements) or incorporated into a food item.

As used herein, lignan or another compound noted above is “purified” or “isolated” from its natural source by removing the natural source from its original environment and then wholly or partially isolating the lignan or other compound from the components with which it is associated in the natural source. Thus, even when “purified”, the compound may be associated with other components originally existing in the natural source. In the case of a natural source that contains a large amount of the compound, only crude purification may be necessary to suitably purify the compound. For instance, harvesting and crushing of flax seeds to produce flax flour may be all the purification that is necessary to purify lignan in the flax seeds to the desired degree. As used herein, “purified” includes what is commonly understood by the terms “purified” and “concentrated.” The term also is intended to include synthetic forms of the compounds, which are often made in purified form, and need no actual purification per se. For instance, a commercially-available form of tocopherol would not ordinarily need to be further purified before being included in the compositions as described herein. As used herein, “dietary supplement” includes a formulation that is intended to be consumed in addition to a person's normal daily diet.

The lignan and additional compounds can be present in the composition as a mixture. For instance, a composition that includes “tocopherol” can include alpha tocopherol, or can contain a mixture of alpha, beta and gamma tocopherols. Alternatively, the lignan and the additional compounds can be a mixture derived from a number of different sources, for instance, a composition that includes “catechin” can include a mixture of catechins from green tea and chocolate.

In one aspect, the invention provides a composition comprising a lignan, such as a flax lignan, and an additional compound selected from the group consisting of: an isoflavone, a tocopherol, a phytosterol, a polyphenol, a catechin, an anthocyanin, an astaxanthin, a glucosamine, and combinations of any thereof. In one non-limiting embodiment, the composition is formulated to provide a “serving” of about 100 mg to about 500 mg of flax lignan. The non-limiting embodiment also may provide one or more of about 15 mg to about 200 mg tocopherol, about 15 mg to about 120 mg isoflavones, about 50 mg to about 400 mg phytosterols, about 50 mg to about 500 mg polyphenols, about 15 mg to about 300 mg catechins, about 15 mg to about 200 mg anthocyanins, about 0.5 grams to about 2 grams of glucosamine, and about 50 mg to about 500 mg polyphenols per serving. A subject can take a dosage of preferably about 2 servings to about 4 servings per day.

The composition can be formulated with the lignan and the additional compounds present in relative percentages or ratios. For instance, the composition can include lignan and isoflavone, where the lignan present is about 4 times to about 6 times (by weight) the amount of isoflavone present. For a composition including lignan and tocopherol, the amount of lignan present can be about 2 times to about 6 times (by weight) the amount of tocopherol present. For a composition including lignan and phytosterol, the amount of lignan present can be about 1 time to about 2 times (by weight) the amount of phytosterol present. For a composition including lignan and polyphenol, the amount of lignan present can be about 1 time to about 2 times (by weight) the amount of polyphenol present. For a composition including lignan and catechin, the amount of lignan present can be about 1 time to about 6 times (by weight) the amount of catechin present. For a composition including lignan and anthocyanin, the amount of lignan present can be about 2 times to about 6 times (by weight) the amount of anthocyanin present. For a composition including lignan and astaxanthin, the amount of lignan present can be about 100 times to about 400 times (by weight) the amount of astaxanthin present. For a composition including lignan and glucosamine, the amount of glucosamine present can be about 5 times (by weight) the amount of lignan present.

The desired serving or dosage levels of the compounds within the compositions of the present invention may vary according to factors such as, for example, the age, sex, and weight of the person consuming them. Daily dosage levels may be adjusted to provide what is deemed to be optimum levels depending on the intended usage. A basic starting dosage, which can then be adjusted, can be about 2 to about 4 “servings” per day.

By saying that the compositions of the present invention provide a certain amount of a compound per day means that the composition is formed in either single daily dosages that provide that amount, or that the daily dosage amount is divided among multiple servings, with the intention that the user consume the multiple servings within a day to consume the daily dosage amount. The daily dosages herein are intended to be general guidelines, and the person consuming the composition will necessarily have the option of consuming either more or less.

The lignans and other compounds used to make the compositions herein can be obtained from either natural or synthetic sources. For instance, a composition containing lignan and tocopherol can be made from the combination of crushed flax seed and synthetic vitamin E. The composition can also contain a single compound obtained from multiple sources. For example, tocopherol within a composition can be a mixture of natural and synthetic vitamin E.

The compounds can be included in the compositions herein in a variety of forms such as, for example, highly purified form or crude form. The catechins, for instance, are commonly found in cocoa and green tea. Catechins can therefore be added to the compositions in the form of a green tea extract. Alternatively, catechins can be added to the compositions in the form of a chocolate coating on a food product, where the amount of chocolate included is calculated to add the desired amount of catechins. The sources can be readily chosen by one of ordinary skill in the field, on the basis of, for example, cost, bioavailability, convenience in formulation, and taste. For instance, the flax lignans can be included in the composition in the form of flax fiber, or as flax oil. Flax fiber can contain about 8 mg lignan per 1650 mg of fiber, while flax oil can contain 12 mg lignan per 15 ml oil.

The compositions of the present invention can be formulated as a tablet, liquid, powder, or any other suitable form. The tablets are intended to be taken orally as a dietary supplement, but can also be produced in forms that could be dissolved in liquids or crushed and consumed as a particulate, either by itself or mixed with food. The tablets can be in the form of, for example, compressed powders, or in the form of gel capsules. The lignan content of flax oil is generally higher than that of flax fiber, and it may therefore be more convenient to provide the lignans in the form of gel capsules containing flax oil and any additional compounds.

The compositions of the present invention also can be produced in liquid form, with the intention that a particular volume be consumed, or the compositions can be produced as powders. Consumers can mix the powders into other products, such as beverages including, but not limited to, juices, milks, soy milks, infant formulas, sports drinks, energy drinks, health drinks, and other suitable beverages. For instance, the compositions can be delivered in powder on a per “unit” basis, where one unit (e.g., one measured spoonful) provides the desired a single serving or the daily dosage of the compounds within the composition.

The compositions may further include natural and/or artificial flavoring components, dyes or other coloring additives, preservatives, inert excipients, and other conventional food supplement additives known in the art. Such ingredients can include ingredients added for reasons of flavor, palatability, color, texture, bulk, meltability, dissolvability, or time-release functionality.

In all forms, the compositions can be included in food products, for instance, into crackers, cheese, yogurt, crisps, cereals, chips, peanut butter, cookies, ice cream, pretzels, gelatin containing snacks, puddings, rice or other grain cakes, food bars, e.g., snack or meal bars, or as additives in processed foods. For example, the compositions may be provided in powdered forms and mixed into dough for baked goods.

The compositions can also be used as a course of treating a disease or condition. In one embodiment, a marker specific for the disease or condition is assayed in a person, the person takes the composition for a period of time, and the marker is assayed again. A reduction (or increase, as is appropriate for the marker) in the level of the marker indicates that the disease is being treated. For instance, appropriate markers for heart disease include, but are not limited to, serum LDL cholesterol, c-Reactive Protein, a soluble adhesion molecule and serum oxidized LDL cholesterol. For diabetes, appropriate markers include, but are not limited to, HBA1c, fasting serum insulin, fasting serum glucose, postprandial serum insulin and postprandial serum glucose. HbA1c (hemoglobin A1c, glycosylated hemoglobin A1c, glycohemoglobin A1c) is a variety of hemoglobin. The HbA1c test assays average blood glucose over a period of two to three months, by measuring the number of glucose molecules attached to hemoglobin. For prostate disease, appropriate markers include, but are not limited to, Prostate Specific Antigen, peak urine flow, IPSS score. Other behavioral scores can also be used. For osteoporosis, appropriate markers include, but are not limited to, bone mineral content and bone mineral density.

The compositions can also be taken for general health, e.g., to prevent a disease or condition.

The compositions described herein can be made and used in a wide variety of forms, and is preferably a solid dosage form. The solid dosage form of the composition can be a tablet, capsule, caplet, granule, particulate, agglomerate, spansule, chewable tablet, lozenge, pellet, suppository or troche. The compositions of the present invention may occur as pharmaceutical compositions, over-the-counter (OTC) compositions, food supplements, dietary supplements, health foods, medical foods, nutraceuticals, veterinary products and fodders. Preferred dosage forms are tablets and gelatin caplets.

Preferably, the dosage form is provided as a single dose or subdivided into several unit doses containing appropriate quantities of the active ingredients. The unit dosage form can be a packaged preparation, the package containing discrete quantities of preparation, for example, packeted tablets, lozenges, granules or pellets. The unit dosage form is the tablet, capsule, lozenge, granule or pellet itself or it is an appropriate number of any of these in packaged form.

The term “unit dosage form” includes a single or multiple dose form containing the described quantities of the active ingredient and one or more excipients, the quantity being such that one or more predetermined units are normally required for a single beneficial administration. In the case of multiple dose forms, such as scored tablets, the predetermined unit will be one fraction such as a half or quarter of a scored tablet of the multiple dose form. The specific dose level for any patient will depend upon a variety of factors including the indication being treated, patient health, age, sex, weight, diet, and pharmacological response, and other such factors. For convenience, the total daily dosage may be divided and administered in portions during the day if desired or at one time, morning, afternoon, night as well as biphasic, triphasic, etc. Controlled, delayed (e.g., enteric), and sustained release formulations are within the scope of the invention and, for convenience, are termed “controlled release” formulations.

The components of the solid dosage form are preferably finely divided, that is, powdered or granulated so as to provide a uniform distribution of ingredients throughout the dosage form. Finely divided components also flow well in tablet presses and other processing machinery, and tend to make tablets having advantageous properties, including, but not limited to, chip resistance, homogeneity, etc.

The active ingredients used in the compositions of the invention can be provided in essentially any commercially available form. They can, for example, be provided as an agglomerate. They can also be provided in either a coated form or a non-coated form. Materials which can be used to coat these active ingredients include, by way of example, gelatin, mono- and di-glycerides (preferably mono- and di-glycerides of edible fatty acids such as, but not limited to, DESCOTET™ or ROCOAT™), stearic acid, a cellulose polymer (such as, but not limited to, carboxymethylcellulose, methylcellulose, hydroxypropylcellulose, hydroxypropylmethylcellulose, ethylcellulose, etc.), corn protein, cellulose acetate phthalate, or similar coating agents. The choice of coating agent is not critical and the selection of such agents is within the knowledge of the skilled artisan. One or more of the active ingredients can be granulated, using either a wet granulation procedure or a dry granulation procedure, in order to improve their processibility. The active ingredients can be agglomerated either separately or in combination and can be agglomerated with one or more excipients, e.g., to form an agglomerate comprising at least about 25% by weight of a carbohydrate based material, about 1-10% by weight of a water soluble binder, and the remaining weight of active ingredient(s).

Ingredients to be incorporated in the tablet can be pretreated to form granules. This process is known as granulation. As commonly defined, “granulation” includes any process of size enlargement whereby small particles are gathered together into larger, permanent aggregates to yield a free-flowing composition having a suitable consistency. Such granulated compositions may have consistency similar to that of dry sand. Granulation may be accomplished by agitation in mixing equipment or by compaction, extrusion, or agglomeration. Any wet or dry granulation method known in the art or hereafter developed can be used to granulate the components described herein; the precise method used is not critical. For example, in a dry granulation method, dry ingredients (e.g., an active ingredient and an excipient) are blended to uniformly disperse each in the other. A granulation agent can be added, to which the dry ingredients adhere. Adherence of the ingredients to the granulation agent generates larger, uniform particles that have advantageous handling properties. In an example of a wet granulation method, dry ingredients are blended to uniformly disperse each in the other(s). A granulating solution (i.e., a binding agent in solution) is added to the blended dry ingredients, and the binding agent binds the ingredients. The mixture is dried and optionally milled. The resulting product comprises particles that also have advantageous handling properties.

Ingredients to be incorporated into the solid dosage form can also be pretreated to form agglomerates. A process for making an agglomerate generally comprises the steps of forming a fluidized bed of carbohydrate particles, intermittently spraying a solution of the water soluble binder in droplet form into the fluidized bed so as to cause intimate mixing of solution and carbohydrate particles and adhesion together of carbohydrate particles to form agglomerated particles, drying the particles in the fluidized bed between intermittent sprayings, and continuing spraying and drying until the desired amount of solution has been sprayed into the bed.

Thereafter, the agglomerated particles are dried to a desired moisture content or the equilibrium moisture content. The carbohydrate-based agglomerate and an active ingredient can be mixed, preferably in a low shear blender. Lubricants, flavors, and other ingredients can also be mixed into the agglomerate with the active ingredient.

Preferred agglomerates include those comprising the following materials: dextrose monohydrate; dextrose monohydrate and maltodextrin; fructose; dextrose; mannitol; fructose and maltodextrin; sucrose; sucrose and maltodextrin; lactose; lactose and maltodextrin; maltose; maltose and maltodextrin; xylose; xylose and maltodextrin. Aqueous solutions of the following materials can be used as a liquid binder solution: corn syrup solids; dextrose; sucrose; poly(vinylpyrrolidone); cooked starch paste; and combinations of the foregoing, any of which may also include maltodextrin.

One or more of the ingredients can also be coated before being incorporated into the solid dosage form. Ingredients that have been coated, granulated, or agglomerated individually or in combination can be further coated, agglomerated or granulated prior to being compressed into a solid dosage form.

A compressed tablet is generally made by mixing the active ingredients with one or more excipients to form a mixture which is subsequently compressed into a tablet. The tablet is optionally coated. Active ingredients and excipients can be individually granulated, using either a wet or dry granulation process, or two or more of these can be mixed prior to granulation. As is known in the art, granulation of ingredients can improve their handling and processing properties including, but not limited to, tendency to flow, ease of mixing with powders, agglomerates, or other granulated products, etc.

In a preferred form, the active ingredients are mixed with the one or more excipients and compressed to form a tablet. The tablet is then optionally coated with one or more coats. The core of the tablet and its coating and outer layers (if any) may include, in addition to the active ingredient(s), excipients such as fillers, binders, disintegrants, lubricants, glidants, surfactants, dyes, and flavorings.

Pharmaceutically acceptable excipients which can be included in the solid dosage form include, for example, binders, acidifying agents, alkalinizing agents, adsorbents, plasticizers, preservatives, antioxidants, buffering agents, colorants, dispersants, thickeners, solubilizing agents, encapsulating agents, stiffening agents, anti-adherents, diluents, coating agents, disintegrants, glidants, surfactants, lubricants, opaquants, polishing agents, dyes, pigments, fillers, flavorants and sweetening agents.

The phrase “pharmaceutically acceptable” includes those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Fillers for use in the tablets of the invention will be those fillers known to those skilled in the art of tablet formulation. Such fillers may be soluble or insoluble and swelling or non-swelling and include, for example, microcrystalline cellulose, dibasic calcium phosphate, tribasic calcium phosphate, calcium carbonate, calcium sulphate, dextrose, kaolin, lactose, powdered cellulose, pregelatinised starch, starch, sucrose and mixtures thereof.

Lubricants conventional to the art may also be used in the outer layer, such as magnesium stearate, zinc stearate, calcium stearate, stearic acid, sodium stearyl fumerate, hydrogenated vegetable oils, glyceryl palmitostearate, glyceryl behenate, sodium benzoate, sodium lauryl sulphate, magnesium lauryl sulphate, mineral oil, talc and mixtures thereof.

As used herein, the term “coating agent” includes a compound used to coat a formed solid dosage form for the purpose of protecting against active ingredient decomposition by atmospheric oxygen or humidity, to provide a desired release pattern for the active ingredient after administration, to mask the taste or odor of the active ingredient substance, or for aesthetic purposes. The coating may be of various types, including sugar coating, film coating, or enteric coating. Sugar coating is water-based and results in a thickened covering around a formed tablet. Sugar-coated tablets generally dissolve at the higher pH values of the intestines. A film coat is a thin cover around a formed tablet or bead. Unless it is an enteric coat, the film coat will dissolve in the stomach. An enteric coated tablet or bead will pass through the stomach and break up in the intestines. Film coatings such as those described above are included within this definition.

The outer coating or layer can include glidants conventional to the art such as colloidal silicon dioxide; disintegrants conventional to the art, such as carboxymethylcellulose calcium, carboxymethylcellulose sodium, magnesium aluminium silicate, microcrystalline cellulose, polacrilin potassium, pregelatinized starch, sodium alginate, sodium starch glycolate, and mixtures thereof.

Surfactants can also be included in the outer coating. Surfactants conventional to the art can be anionic (e.g., sodium lauryl sulphate), cationic or neutral surfactants ionic salts (e.g., sodium chloride).

Dyes, pigments and flavorings conventionally used in the art of tablet formulation can also be included in the composition.

These formulations can also contain hygroscopic agents which can draw water into the tablet. Such hygroscopic agents can include water soluble electrolytes, small organic compounds, osmotic adjusting agents to increase the osmotic pressure within a dosage form and attract water.

The dosage form of the invention can contain any of a variety of hydrophobic or hydrophilic binders. Examples of suitable hydrophobic binders include cellulose acetate butyrate, cellulose acetate propionate, cellulose propionate high molecular weight (200,000), cellulose propionate medium molecular weight (75,000), cellulose propionate low molecular weight (25,000), cellulose acetate, cellulose nitrate, ethylcellulose, polyvinyl acetate, and others known to those of ordinary skill in the art. Examples of suitable hydrophilic binders include poly(vinylpyrrolidone), vinyl alcohol polymer, polyethylene oxide, water soluble or water swellable cellulose and starch derivatives and others known to those of ordinary skill in the art.

Examples of other binders which can be added to the formulation include, for example, acacia, tragacanth, gelatin, starch, cellulose materials such as ethyl cellulose, methyl cellulose, hydroxypropyl methylcellulose, and sodium carboxymethyl cellulose, alginic acids and salts thereof, polyethylene glycol, guar gum, polyvinylpyrrolidone, polysaccharide, sugars, invert sugars, poloxomers (including, but not limited to, PLURONIC™ F68, PLURONIC™ F127), collagen, albumin, gelatin, cellulosics in non-aqueous solvents, pre-gelatinized starch, starch paste and combinations of the above. Other binders include, for example, polypropylene glycol, polyoxyethylene-polypropylene copolymer, polyethylene ester, polyethylene glycol, polyethylene sorbitan ester, polyethylene oxide or combinations thereof and others known to those of ordinary skill in the art.

Disintegrants include materials which aid in the disintegration and/or dissolution of the solid dosage form and/or its ingredients. Disintegrants include starches such as, but not limited to, corn starch, potato starch, pre-gelatinized and modified starches thereof; cellulosic agents such as, but not limited to, Ac-di-sol, montmorrilonite clays, cross-linked PVP, sweeteners, bentonite, VEEGUM™, microcrystalline cellulose, alginates, sodium starch glycolate; gums such as, but not limited to, agar, guar, locust bean, karaya, pectin and tragacanth.

Plasticizers may be required in the solid dosage form of the invention. Such plasticizers can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate esters, triacetin, propylene glycol phthalate esters, phosphate esters, sebacate esters, glycol derivatives, fatty acid esters, glycerin, ethylene glycol, 1,2-butylene glycol, 2,3-butylene glycol, styrene glycol, diethylene glycol, dipropylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, dimethylsebacate, di-2-ethylhexylsebacate, tricresyl phosphate, triethyl phosphate, triphenyl phosphate, acetylated monoglycerides, mineral oil, castor oil, glyceryl triacetate, butyl stearate, glycerol monostearate, butoxyethyl stearate, stearyl alcohol, cyclohexyl ethyl phthalate, cyclohexyl methyl dibutylphthalate, diethyl phthalate, dibutyl phthalate, diisopropyl phthalate, dimethyl phthalate, dioctyl phthalate, acetyl tributyl citrate, triethyl citrate, acetyl triethyl citrate, tributyl citrate, allyl glycolate and combinations thereof. All such plasticizers are commercially available from sources such as Aldrich or Sigma Chemical Co. or Morflex, Inc.

As used herein, the term “acidifying agent” includes a compound used to provide acidic medium for product stability. Such compounds include, by way of example and without limitation, acetic acid, citric acid, fumaric acid, hydrochloric acid, and nitric acid and others known to those of ordinary skill in the art.

As used herein, the term “alkalinizing agent” includes a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium hydroxide, triethanolamine, and trolamine and others known to those of ordinary skill in the art.

As used herein, the term “adsorbent” includes an agent capable of holding other molecules onto its surface by physical or chemical (chemisorption) means. Such compounds include, by way of example and without limitation, powdered and activated charcoal and others known to those of ordinary skill in the art.

As used herein, the term “preservative” includes a compound used to prevent the growth of microorganisms or prevent the degradation of one or more active ingredients. Such compounds include, by way of example and without limitation, benzalkonium chloride, benzethonium chloride, benzyl alcohol, cetylpyridinium chloride, chlorobutanol, phenol, phenylethyl alcohol, phenylmercuric nitrate and thimerosal and others known to those of ordinary skill in the art.

As used herein, the term “antioxidant” includes an agent which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and others known to those of ordinary skill in the art.

As used herein, the term “buffering agent” includes a compound used to resist change in pH upon dilution or addition of acid or alkali. Such compounds include, by way of example and without limitation, potassium metaphosphate, potassium phosphate, monobasic sodium acetate and sodium citrate anhydrous and dihydrate and others known to those of ordinary skill in the art.

As used herein, the term “colorant” includes a compound used to impart color to solid pharmaceutical preparations (e.g., tablets and capsules). Such compounds include, by way of example and without limitation, FD&C Red No. 3, FD&C Red No. 20, FD&C Yellow No. 6, FD&C Blue No. 2, FD&C Green No. 5, FD&C Orange No. 5, FD&C Red No. 8, caramel, and ferric oxide, red and others known to those of ordinary skill in the art. Coloring agents can also include pigments, dyes, tints, titanium dioxide, natural coloring agents such as grape skin extract, beet red powder, beta carotene, annato, carmine, turmeric, paprika, CHROMAKOTE™ and others known to those of ordinary skill in the art.

As used herein, the term “flavorant” includes a natural or artificial compound, or some combination of these, used to impart a pleasant flavor and often odor to a pharmaceutical preparation. Flavors incorporated in the composition may be chosen from natural and synthetic flavor oils and flavoring aromatics and/or natural oils, extracts from plants, leaves, flowers, fruits, and combinations thereof. Such compounds include, by way of example and without limitation, anise oil, cinnamon oil, vanilla, vanillin, cocoa, chocolate, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils such as lemon, orange, lime and grapefruit oils; and fruit essences, including berry, apple, pear, peach, date, blueberry, kiwi, strawberry, raspberry, wildberry, cherry, plum, pineapple, and apricot. All of these flavorants are commercially available. Preferred flavorants include vanillin and berry. The amount of flavoring may depend on a number of factors, including the organoleptic effect desired.

As used herein, the term “sweetening agent” includes a compound used to impart sweetness to a preparation. Such compounds include, by way of example and without limitation, aspartame, dextrose, glycerin, mannitol, saccharin sodium, sorbitol, sucrose high fructose corn syrup, fructose oligosaccharides, and others known to those of ordinary skill in the art.

As used herein, the term “tablet anti-adherents” includes agents which prevent the sticking of ingredients to punches and dies in a tabletting machine during production. Such compounds include, by way of example and without limitation, magnesium stearate, corn starch, silicone dioxide, talc and others known to those of ordinary skill in the art.

As used herein, the term “tablet binders” includes substances used to cause adhesion of powder particles in tablet granulations. Such compounds include, by way of example and without limitation, acacia, alginic acid, carboxymethylcellulose sodium, compressible sugar (e.g., NUTAB™), ethylcellulose, gelatin, liquid glucose, methylcellulose, povidone, pre-gelatinized starch and others known to those of ordinary skill in the art.

As used herein, the term “tablet and capsule diluent” includes inert substances used as fillers to create the desired bulk, flow properties, and compression characteristics in the preparation of tablets and capsules. Such compounds include, by way of example and without limitation, dibasic calcium phosphate, kaolin clay, fructose, sucrose, dextrose, lactose, mannitol, microcrystalline cellulose, powdered cellulose, precipitated calcium carbonate, sorbitol, calcium sulfate, starch and others known to those of ordinary skill in the art.

As used herein, the term “direct compression excipient” includes a compound used in direct compression tablet formulations. Such compounds include, by way of example and without limitation, dibasic calcium phosphate (e.g., DITAB™), spray dried, or anhydrous lactose, microcrystalline cellulose, (AVICEL™), dextran (EMDEX™), sucrose (NUTAB™) and others known to those of ordinary skill in the art.

As used herein, the term “glidant” includes agents used in tablet and capsule formulations to reduce friction during tablet compression. Such compounds include, by way of example and without limitation, colloidal or fumed silica, magnesium stearate, comstarch, and talc and others known to those of ordinary skill in the art.

As used herein, the term “lubricant” includes substances used in tablet formulations to reduce friction during tablet compression. Such compounds include, by way of example and without limitation, calcium stearate, magnesium stearate, mineral oil, stearic acid, hydrogenated vegetable oil, benzoic acid, poly(ethylene glycol), NaCl, PRUV™, zinc stearate and others known to those of ordinary skill in the art.

As used herein, the term “tablet/capsule opaquant” includes a compound used to render a capsule or a tablet coating opaque. Opaquants can be used alone or in combination with a colorant. Such compounds include, by way of example and without limitation, titanium dioxide and others known to those of ordinary skill in the art.

As used herein, the term “polishing agent” includes a compound used to impart an attractive sheen to coated tablets. Such compounds include, by way of example and without limitation, carnauba wax, and white wax and others known to those of ordinary skill in the art.

The solid dosage forms, or one or more of the ingredients, can also be coated. Coating an ingredient can mask an unpleasant or non-desired taste or odor associated with the component. In addition, coating can also stabilize a component, particularly where the component which can lose its physiological activity, or have such activity decreased or inhibited, upon exposure (particularly prolonged exposure) to an environmental factor such as light, oxygen, or moisture. Coating of unpleasant-tasting or -smelling components is contemplated when the dosage described herein is a chewable or quickly-dissolving composition. When the dosage is a tablet, coating of individual components is generally not necessary, although coating of the tablet can serve to improve the stability, appearance, taste, odor, or handling characteristics of the tablet.

The pressed solid dosage form of the invention can comprise a film coating and a compressed solid core. The film coating comprises one or more film forming agents, e.g., combinations of film forming agents are used in some embodiments of the film coating. This combination of film forming agents can provide a formulation having a combined delayed and controlled release of therapeutic agent; however, an immediate release dosage form is preferred. The film coating on the dosage form can also comprise a flavorant and/or colorant, such as a pigment or dye. The coating for the pressed tablet is preferably a rapidly dissolving finish or polish coat comprising, for example, a cellulosic polymer, a colorant, a flavorant and a wax.

The terms “film forming agent” includes polymeric compounds (of natural, synthetic, semi-synthetic or genetically engineered sources) which will form a film coating around the solid core of the formulation.

A film coating employed can comprise a polymer with a pH dependent solubility which would release a major portion of one or more ingredients in the stomach, ileum, jejunum, small intestine or large intestine a person taking the tablet. The thickness of the film coating can be varied as desired.

A film coating may be prepared by applying a solution, suspension or emulsion to an existing core or solid and removing the liquid portion to form a substantially dry film. The film coating can include one or more of the following by way of example and without limitation: cellulose acetate, ethyl cellulose, wax, EUDRAGIT™ E100, EUDRAGIT™ RS, and EUDRAGIT™ RL, EUDRAGIT™ L, EUDRAGIT™ S, cellulose acetate phthalate, hydroxypropyl methylcellulose phthalate, HPMC acetate succinate, cellulose acetate butyrate, cellulose acetate propionate, cellulose propionate, HPMC, carrageenan, cellulose nitrate, hydrophilic cellulosic agents, hydroxypropylcellulose, methylcellulose, hydroxyethylcellulose, ethylcellulose, polyvinyl acetate and latex dispersions, poly-acids, enteric polymers, polysaccharides, acacia, tragacanth, guar gum, gelatin, proteins, albumin, polylactic acid, biodegradable polymers, polyglutamic acid, carnauba wax, DRI KLEAR™ (Crompton & Knowles, cellulose based polymer dispersion), CHROMAKOTE™ (Crompton & Knowles, pigmented dispersion) and combinations thereof.

The film coating can also include, by way of example and without limitation, poly(ethylene glycol) 3350 (PEG 3350), sorbitol, sucrose, polyols, xylitol, mannitol, carbohydrates, sugars, lactose, maltose, dextrose, water soluble cyclodextrins, urea, fructose, sucrose, mannose; a-hydroxy acids such as citric acid, tartaric acid, fumaric acid, succinic acid, glycolic acid, lactic acid, combinations thereof and their salts; halide counter-ions such as bromide, fluoride, iodide and chloride; divalent metal cations such as magnesium and calcium; anionic agents such as phosphates, sulfates, sulfonates, nitrates, bicarbonates, combinations thereof and their salts; cellulosics such as HPC; poly(ethylene oxide); poly(vinyl pyrrolidone); gums and gelling agents such as xanthan gum, alginic acid, thereof and their salts; clays such as montmorillonite clay, bentonite, Veegum, kaolin clay; miscellaneous ones such as kieselguhr, magnesium silicate, bentone, hectorite, PLURONICS™, hydrophilic surfactants; polyols such as sorbitol, mannitol, xylitol; proteins such as collagen; water soluble amino acids; disintegrants such as starch, sodium starch glycolate, croscarmellose; and water soluble organic compounds; and combinations thereof.

In compositions having an outer polymer layer, the layer can include, by way of example and without limitation, a pH independent hydrophilic polymer (such as, but not limited to, hydroxypropylmethylcellulose), one or more fillers (such as, but not limited to, microcrystalline cellulose, dibasic calcium phosphate), a lubricant (such as, but not limited to, sodium stearyl fumerate), and a glidant (such as, but not limited to, colloidal silicon dioxide). The outer layer can include a pH independent hydrophilic polymer, such as, but not limited to, hydroxypropylmethyl cellulose.

Chewable tablets and powdered dietary supplements are well known in the nutritional products industry. These products are intended to provide a nutritious and bioavailable product while at the same time providing a product with good palatability, or organoleptic effect. Generally, however, the more vitamins and minerals a product contains the less palatable the product is. Taste-masking technology has been developed for preparation of palatable chewable tablets. This technology generally requires encapsulation of the ingredients in the tablet or addition of sugars, sweeteners, acceptably flavored agents, or some combination of these, to the composition before it is formulated into a tablet.

When the tablet is a chewable tablet, it will preferably comprise a carbohydrate based agglomerate, and it will preferably have an interior that is detectably (e.g., by members of a taste panel or by patients) softer than its exterior. This type of construction in combination with the agglomerate facilitates the rapid dissolution of the tablet in the mouth shortly after it has been chewed. Further, this type of construction reduces the tablet's extent of atmospheric water absorption, i.e., it has reduced hygroscopicity.

An exemplary process for making a chewable tablet from a carbohydrate-based agglomerate comprises blending the agglomerate with active ingredients and a lubricant to form a substantially homogeneous mixture, which is placed in a conventional tablet-forming apparatus and compressed to a hardness sufficient to hold the tablet together and substantially destroy the open pore structure of the agglomerate at the surface of the tablet while substantially maintaining the open pore, i.e., large surface area, structure of the agglomerate in the interior of the tablet. Thus, the agglomerate is compressed so that the interior of the tablet retains the essential porous structure and other physical characteristics of the agglomerate which enable it to liquefy quickly when chewed, while the physical characteristics of the agglomerate are changed primarily at the surface of the tablet.

One or more excipients are included in the chewable tablet of the invention. An excipient includes, without limitation, an acidifying agent, alkalinizing agent, adsorbent, antifungal preservative, antioxidant, buffering agent, colorant, disintegrant, encapsulating agent, flavorant, hygroscopic agent, plasticizer, stiffening agent, sweetening agent, tablet anti-adherent, tablet binder, tablet and capsule diluent, tablet coating agent, tablet direct compression excipient, tablet disintegrant, tablet glidant, tablet lubricant, tablet/capsule opaquant and tablet polishing agent.

A capsule is generally made by mixing the active ingredients (which can be powdered, granulated, coated, agglomerated, or some combination of these) with one or more excipients to form a mixture which is subsequently loaded into the capsule. The capsule is preferably a hard gelatin capsule. The capsule halves are then joined.

Various forms of extended release particles or coatings along with immediate release particles or coatings can also be combined in the present formulations to deliver the various ingredients at various rates. Formulations having a combination of particles with different release profiles are well known and are prepared according to procedures and techniques known to the artisan of ordinary skill.

In one aspect, the invention includes a rapid-release tablet composition, which can include an inner core comprising the active ingredient and a rapidly disintegrating outer coating or layer, to provide a rapid release of the active ingredient. When a layer or coating is stated to disintegrate “rapidly”, this means that it disintegrates over a time period of, for example, less than 30 minutes, for example less than 10 minutes after administration.

The invention can include a delayed release composition, which can include an inner core comprising the active ingredient and an outer layer or coating that does not disintegrate rapidly, e.g., which includes comprising a pH independent hydrophilic polymer together with one or more fillers. The outer layer is gradually removed by a combination of dissolution and erosion following administration and the inner core disintegrates rapidly once exposed. When a layer or coating is “gradually” removed, this means that it is removed over a time period of, for example, 1-8 hours, such as 1-3.5 hours, 2-5 hours or 4-6 hours following administration.

The term “pH independent hydrophilic polymer” will be well-understood by those skilled in the art. Such polymers dissolve/erode after administration at a rate which is independent of the pH of the surrounding fluid. Such polymers include, for example, cellulose ethers, polyvinylpyrrolidone, mixtures of natural hydrophilic gums, e.g., guar gum, gum Karaya, tragacanth and xanthan gum, and mixtures thereof. Preferably cellulose ethers will be employed, most preferably hydroxypropylmethylcellulose.

A “rapidly dissolving” ingredient is an ingredient wherein a known amount of the compound will substantially saturate or completely dissolve in a sufficient volume of saliva or a gastrointestinal fluid in less than about 3 hours, preferably less than about 1 hour.

A “slowly dissolving” ingredient is an ingredient wherein an amount of the compound will substantially completely dissolve in a sufficient volume of saliva or a gastrointestinal fluid in greater than about 3 hours, preferably greater than about 8 hours, more preferably greater than about 12 hours, and even as long as 48 hours (but preferably not greater than 24 hours).

One form of the solid dosage form comprises ingredients which have an immediate release profile and ingredients which have a controlled release profile. It will be understood by those of ordinary skill that a “controlled release” dosage form includes dosage forms providing a sustained release, extended release, and timed release.

In rapid-release forms of the invention, a rapidly disintegrating inner core and a rapidly disintegrating outer coating may have the same composition, which can include one or more fillers (e.g., microcrystalline cellulose, lactose), binders (e.g., polyvinylpyrrolidone, pregelatinised starch), disintegrants (e.g., microcrystalline cellulose, pregelatinised starch) and lubricants (e.g., sodium stearyl fumerate, magnesium stearate).

The compositions of the invention can have an enteric coating, which will delay the initiation of the erosion/disintegration of the underlying layer until the tablet reaches a region of the gastrointestinal tract where a specific pH prevails. Such enteric coated tablets allow targeting of the active ingredient to the colon. Enteric coatings for use in the tablets of the invention will be those coatings known to those skilled in the art. Such coatings include cellulose acetate phthalate, polyvinyl acetate phthalate, shellac, styrene maleic acid copolymers, methacrylic acid copolymers and hydroxypropyl methylcellulose phthalate. When the pharmaceutical compositions of the invention are enterically coated they are particularly useful for treating diseases of the lower gastrointestinal tract.

The compositions according to the invention may be prepared according to conventional methods known in the art using conventional tabletting machinery. For example, a pH independent hydrophilic polymer may be blended with one or more fillers, and optionally other excipients, and compressed onto a core of one or more inner layers each comprising an active ingredient. Cores of active substance may be prepared, for example, by compression of material produced by dry slugging, wet granulation or dry blending. Blends for rapidly disintegrating outer coatings may be prepared in the same way and compressed onto the pH independent hydrophilic polymer coated cores.

The direct dry compression tabletting composition or formulation is typically prepared by first dry mixing all or a portion of the ingredients in a high shear pharmaceutical type mixer, such as a Zanchetta Roto-G, a Littleford MGT, a Baker-Perkins High Shear, a Littleford FKM mixer, etc., to form a first, dry blended mixture. The remaining ingredients are added and the mixture blended.

It will be apparent obtaining acceptable tablets will depend on the moisture content and other characteristics of the mixture, which will depend on many factors such as the moisture content of the ingredients, the temperature and relative humidity of the air in the plant where the production operations are carried out, the overall processing duration and the particle size of the final product.

The mixture can be dried, e.g., it can be allowed to stand until dried, or it can be fed into a continuous drier or batch drier such as a turbotray drier, direct-heat rotary drier, drum drier, belt drier, spray drier, fluid bed drier and similar industrial driers well known to those skilled in chemical technology (see, e.g., “Drying” in Kirk-Othmer's Encyclopedia of Chemical Technology, vol. 8, pages 91-112, 1979). Alternatively, the dehydration of the mixture can be carried out by treating it with a very small volume of a pharmaceutically-acceptable and non-toxic volatile, water-miscible solvent in which the ingredients are insoluble, such as, e.g., acetone.

The final mixture is typically directly tabletted using a conventional tabletting apparatus, e.g., a Manesty Rotary Press, a Stokes Rotary Press, etc. Hardness of the resulting tablets is measured, e.g., in the Schlesinger Hardness Tester. Friability is measured, e.g., with a Eurika® Tab Friability tester, e.g., for 20 tablets after 130 revolutions. The ingredients and tabletting procedures are adjusted until tablets with the desired hardness and friability are produced. Disintegration time and dissolution time are measured using the test methods set forth in U.S. Pharmacoepia XXI. Reference may be made to REMINGTON'S PHARMACEUTICAL SCIENCES (17th ed. 1985) for guidance.

Capsules or “caplets” allow water insoluble ingredients can be encapsulated in a soft gelatin shell. The soft gelatin capsules generally enclose a fluid or semi-fluid fill in which an active ingredient is dissolved or dispersed. Typical fills or fillers for soft gelatin capsules comprise predominantly of triglyceride (triacylglycerol or TAG) oils in which the active ingredient is dissolved. TAG oil is typically a vegetable oil such as, but not limited to, corn oil, peanut oil, safflower oil, sunflower oils, and soybean oil. More specifically, TAG oils comprise three fatty acids attached to a glycerol backbone. Oil is used as the vehicle for the active ingredients because water-based vehicles would dissolve and rupture the integrity of the soft gelatin capsule. Soft gelatin capsules are intended to rupture on contact with water, such as typically found in a patient's gastrointestinal tract.

Soft gelatin encapsulation of a wide range of products is long-established. The basic technique is described in U.S. Pat. No. 2,234,479. Soft gelatin encapsulation methods are further described by U.S. Pat. Nos. 2,349,430; 2,387,747; 2,449,139; 3,592,945; 3,656,997; 4,028,024; 4,670,287; 4,816,259; 4,935,243; 5,146,758; 5,735,105; and 6,656,500.

Soft gelatin capsules generally enclose a fluid or semi-fluid fill material or “filler” in which the active ingredient is dissolved, dispersed and/or otherwise distributed. The manufacture of these capsules involve filling water-soluble shells with mixtures of capsulated material which is generally insoluble in water. In the manufacture of soft gelatin capsules, interaction between water-soluble parts of the capsules and water or aqueous materials typically is minimized or inhibited. The moisture content of the shell is typically maintained at a minimum and the shell is rapidly dried in order to remove as much of the moisture from the shell as possible in the shortest time possible immediately after manufacture and before complete hardening of the capsule when interaction between the shell and the filler or medicament is most likely to take place. The water content of the shell is highest prior to drying at which time water soluble constituents of the filler can migrate into the shell. Thus, it is highly desirable to use water-insoluble fillers such as oils. After the shell has dried and hardened, the capsule is substantially impervious to substances generally carried in the shell and interaction between the shell and the filling stops.

Soft gelatin capsules are typically made using soft gelatin that is highly flexible and deformable. The gelatin is provided as a ribbon prior to encapsulation. The shells are made in any suitable way well-known in the art, for example as described in the listed patents above. The shells of the capsules are typically water-soluble and contain ingredients, such as, but not limited to, water, gelatin, glycerin, hydrogenated starch and starch hydrolysate. The soft gelatin capsule is plasticized by the presence of water as well as by the addition of glycerin, sorbitol, or a similar polyol. The gelatin may be blended with other components to vary its characteristics for different applications. The term “gelatin” includes any gelatin-based composition which is suitable for use in an encapsulation process.

The soft gelatin capsules are typically filled with a filler material or medicament while they are formed and just prior to being sealed. The fillers are virtually insoluble in water in order to prevent interaction between the filling and the shell. Encapsulation machinery typically draws two gelatin ribbons to a charging station where sections of the gelatin strips are sealed around the capsule contents (filler). Encapsulation is normally accomplished using a flat or a roller die technique. In a non-limiting example, the gelatin ribbons are cast on respective casting drums and are then brought together face-to-face between a pair of rotary dies where capsules are formed and filled by an injection wedge. Removal of the capsules from the remaining ribbons is assisted by stripper rollers. In another non-limiting example, the encapsulation machine is of the rotary die type, fed by two receivers, one contains the molten gelatin mass used to form the shell, while the other contains the fill formulation.

In yet another example of the encapsulation process, molten gelatin flows by gravity through heated tubes to two heated spreader boxes. The spreader boxes simultaneously cast the gelatin mass into two ribbons. These are lubricated with a blend of fractionated coconut oil/lecithin and delivered to the rotary dies. The filler flows by gravity into a hopper, which serves as a reservoir to the input of the encapsulation pump. The fill formulation is delivered to the filling point by the positive displacement piston pump. The two gelatin ribbons are fed in between the two rotating dies. The dies contain paired pockets, which form the shape of the soft gelatin capsule and provide the sealing mechanism. At the precise moment that the two die pockets line up, the fill formulation is injected through an encapsulation wedge in between the gelatin ribbons. The seal forms as a result of the pressure between dies and heat applied by the encapsulation wedge to produce the soft gelatin capsule.

Soft gelatin capsules are typically dried by a two phase process. First, capsules are moved to a rotary drier where they are tumbled in warm, low humidity, forced air environment for a predetermined length of time. The second phase begins after discharge from the rotary drier, the capsules are spread in a monolayer on shallow drying trays and low humidity air passed over them. Transfer of water to and from the shell occurs over several days until the water put into the gelatin during gelatin mass production has evaporated. Capsule hardness determinations are performed to monitor the drying process. The capsules are monitored until the hardness is within the specified range. The capsules are then placed into deep holding trays. Capsules are inspected and polished with V.M. & P Naptha to remove the lubricating film on the capsule surface prior to grading and packaging.

In yet another example of soft gelatin capsule manufacturing, U.S. Pat. No. 4,028,024 discloses cooling of newly-extruded capsules during conveyance thereof from an extruder head through the use of cool air blown through a foraminous conveyor belt.

The invention is further illustrated by the following examples, which are non-limiting. The following examples relate specifically to tablets for oral administration, however by altering the shape and formulations of the dosage forms, other solid dosage forms may be had.

### EXAMPLES

### Example 1

### Lignan and Isoflavone Composition in Tablet Form

The compositions can be formulated as tablets. For instance, a tablet containing lignan and isoflavone as described herein can be made in tablet form as described in Table A, below.

TABLE A
Tablet containing lignan and isoflavone.

| Ingredient | Amount | |
|---|---|---|
| Flax lignan concentrate (75 mg SDG; lignan | 215 | mg |
| concentration = 35% SDG) | | |
| Isoflavones (50 mg total isoflavones; for instance, | 125 | mg |
| Novasoy = 40% isoflavones) | | |
| Dicalcium phosphate | 957 | mg |
| Sorbitol | 60 | mg |
| Magnesium stearate | 3 | mg |

The ingredients can be mixed and pressed into tablet form using methods commonly used in the tabletting art. For instance, the ingredients are combined in a commercial mixer until uniformly mixed. The resulting mixture is pressed into tablets using a tablet press.

The tablet may also include dispersion aids, binders (for instance, hydroxypropylcellulose), solvents, bulking agents, flavorings and other ingredients. The tablets may also be coated.

### Example 2

### Lignan and Alpha-Tocopherol Composition in Tablet Form

The compositions can be formulated as tablets. For instance, a tablet containing lignan and tocopherol as described herein can be made in tablet form as described in Table B, below.

TABLE B
Tablet containing lignan and tocopherol.

| Ingredient | Amount | |
|---|---|---|
| Flax lignan concentrate (75 mg SDG; lignan conc = | 215 | mg |
| 35% SDG) | | |
| RRR-alpha-tocopheryl succinate (50 mg RRR-alpha- | 63 | mg |
| tocopherol) | | |
| Dicalcium phosphate | 779 | mg |
| Sorbitol | 60 | mg |
| Magnesium stearate | 3 | mg |

The ingredients can be mixed and pressed into tablet form using methods commonly used in the tabletting art. For instance, the ingredients are combined in a commercial mixer until uniformly mixed. The resulting mixture is pressed into tablets using a tablet press.

The tablet may also include dispersion aids, binders (for instance, hydroxypropylcellulose), solvents, bulking agents, flavorings and other ingredients. The tablets may also be coated.

### Example 3

### Lignan and Isoflavone Composition in Powder Form

The compositions can be formulated as a dry powder. For instance, a powder containing lignan and isoflavone as described herein can be made in powder form as described in Table C, below.

TABLE C
Powder containing lignan and isoflavone.

| Ingredient | Amount | |
|---|---|---|
| Flax lignan concentrate (75 mg SDG; lignan | 215 | mg |
| concentration = 35% SDG) | | |
| Isoflavones (50 mg total isoflavones; for instance, | 125 | mg |
| Novasoy = 40% isoflavones) | | |
| Dicalcium phosphate | 957 | mg |
| Sorbitol | 60 | mg |

The ingredients can be mixed using methods commonly used in formulating dry powders. For instance, the ingredients are combined in a commercial mixer until uniformly mixed. The tablet may also include dispersion aids, solvents, bulking agents, flavorings and other ingredients. The mixture may also be dried, for instance, spray dried, and ground. Preferably the resulting powder is a free-flowing powder.

### Example 4

### Lignan and Alpha-Tocopherol Composition in Powder Form

The compositions can be formulated as a dry powder. For instance, a powder containing lignan and tocopherol as described herein can be made in powder form as described below.

TABLE B
Powder containing lignan and tocopherol.

| Ingredient | Amount | |
|---|---|---|
| Flax lignan concentrate (75 mg SDG; lignan | 215 | mg |
| concentration = 35% SDG) | | |
| RRR-alpha-tocopheryl succinate (50 mg RRR-alpha- | 63 | mg |
| tocopherol) | | |
| Dicalcium phosphate | 779 | mg |
| Sorbitol | 60 | mg |

The ingredients can be mixed using methods commonly used in formulating dry powders. For instance, the ingredients are combined in a commercial mixer until uniformly mixed. The tablet may also include dispersion aids, solvents, bulking agents, flavorings and other ingredients. The mixture may also be dried, for instance, spray dried, and ground. Preferably the resulting powder is a free-flowing powder.

### Example 5

### Lignan and Isoflavone Composition in Coated Tablet Form

The tablets made according to Example 1 above, can be coated. For instance, DRI KLEAR™ and CHROMAKOTE™ can be mixed at a ratio of 2:1 (by weight) together with a flavorant, and the mixture sprayed onto the tablets to form a finish coat. The finish coat can be polished with carnauba wax.

### Example 6

### Lignan Capsules

The lignan and purified compound selected from the group consisting of: an isoflavone, a tocopherol, a phytosterol, a polyphenol, a catechin, an anthocyanin, an astaxanthin, a glucosamine, and combination of any thereof is placed in a capsule or tablet for administration to a subject. The capsule or tablet may be placed in a container such as a bottle associated with indicia or instructions directing a user of a recommended dosage of the lignan and the purified compound selected from the group consisting of: an isoflavone, a tocopherol, a phytosterol, a polyphenol, a catechin, an anthocyanin, an astaxanthin, a glucosamine, and combination of any thereof. A recommended dosage of the lignan may be 860-1720 mg of lignan per day, which is about 300-600 mg of SDG per day.

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition comprising:
a purified lignan; and
a purified compound selected from the group consisting of: an isoflavone, a tocopherol, a phytosterol, a polyphenol, a catechin, an anthocyanin, an astaxanthin, a glucosamine and combinations of any thereof.

2. The composition of claim 1, wherein the lignan is flax lignan.

3. The composition of claim 1, wherein the isoflavone is soy isoflavone.

4. The composition of claim 1, wherein the tocopherol is γ-tocopherol.

5. The composition of claim 1, wherein the glucosamine is glucosamine sulfate.

6. The composition of claim 1, wherein the composition is in the form of a tablet, a powder, or a liquid.

7. A container containing the composition of claim 1, wherein the container is associated with indicia directing a user of a recommended dosage of the composition.

8. The composition of claim 1, where the amount of lignan by weight present in the composition is:
about 4 times to about 6 times the amount of isoflavone by weight present in the composition;
about 2 times to about 6 times the amount of tocopherol by weight present in the composition;
about 1 times to about 2 times the amount of phytosterol by weight present in the composition;
about 1 times to about 2 times the amount of polyphenol by weight present in the composition;
about 1 times to about 6 times the amount of catechin by weight present in the composition;
about 2 times to about 6 times the amount of anthocyanin by weight present in the composition;
about 100 times to about 400 times the amount of astaxanthin by weight present in the composition; or
about ⅕ the amount of glucosamine by weight present in the composition.

9. A dietary supplement comprising:
a purified lignan; and
a purified compound selected from the group consisting of: an isoflavone, a tocopherol, a phytosterol, a polyphenol, a catechin, an anthocyanin, an astaxanthin, a glucosamine and combinations of any thereof.

10. The dietary supplement of claim 9, wherein the lignan is flax lignan.

11. The dietary supplement of claim 9, wherein the isoflavone is soy isoflavone.

12. The dietary supplement of claim 9, wherein the tocopherol is γ-tocopherol.

13. The dietary supplement of claim 9, wherein the glucosamine is glucosamine sulfate.

14. The dietary supplement of claim 9, wherein the dietary supplement is in the form of a tablet, a powder, or a liquid.

15. A container containing the dietary supplement of claim 9, wherein the container is associated with indicia directing a user of a recommended dosage of the dietary supplement.

16. The dietary supplement of claim 9, where the dietary supplement comprises about 100 mg to about 500 mg of lignan, and:
about 15 mg to about 120 mg isoflavone;
about 15 mg to about 200 mg of tocopherol;
about 50 mg to about 400 mg phytosterol;
about 50 mg to about 500 mg polyphenol;
about 15 mg to about 300 mg catechin;
about 15 mg to about 200 mg anthocyanin;
about 0.25 mg to about 5.0 mg astaxanthin; or
about 0.5 grams to about 2 grams glucosamine.

17. A food composition comprising:
a purified lignan; and
a purified compound selected from the group consisting of: an isoflavone, a tocopherol, a phytosterol, a polyphenol, a catechin, an anthocyanin, an astaxanthin, a glucosamine, and combinations of any thereof.

18. The food composition of claim 17, wherein the food product is a food bar, a cookie, a cracker, cheese, yogurt, a crisp, cereal, a chip, peanut butter, ice cream, a pretzel, a gelatin containing snack, pudding, or a rice or other grain cakes.

19. The food composition of claim 17, wherein the food composition is a beverage.

20. The food composition of claim 19, wherein the beverage is selected from the group consisting of juices, milks, soy milks, infant formulas, sports drinks, energy drinks, and health drinks.
